(19) United States
(12) Patent Application Publication
(10) Pub. No.: US 2008/0245362 A1
(43) Pub. Date: Oct. 9, 2008

(54) Nebuliser

(76) Inventors: George Moessis, New South Wales (AU); Jack Vaisman, New South Wales (AU); Philip Jenkinson, New South Wales (AU); David Bull, New South Wales (AU); Andy Wyatt, New South Wales (AU)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 12/066,014

(22) PCT Filed: Sep. 6, 2006
(86) PCT No.: PCT/AU2006/001305

§ 371 Date: Mar. 6, 2008

(30) Foreign Application Priority Data
Sep. 6, 2005 (AU) 2005904861
Sep. 6, 2005 (AU) 2005904881
Sep. 6, 2005 (AU) 2005904937
Feb. 10, 2006 (AU) 2006900670

(51) Int. Cl. A61M 11/06; B05B 1/08; B05B 17/06

(52) U.S. Cl. 128/200.16; 239/338; 239/102.1; 239/4

(57) ABSTRACT

The present invention relates to a nebuliser and method of nebulising a liquid. The nebuliser includes a nebulisation chamber (1) having a well (2) adapted to contain a liquid (3) to be nebulised. An energy source in the form of an ultrasonic transducer (6) has as a curved energy transmission surface (7). This curved energy transmission surface defines a focal point (8) and a focal length (9). The energy source is spaced from the well such that the distance between the focal point and the energy source intrudes into the well not greater than 50% of the focal length. Preferably the well is shaped such that during nebulisation the level of the liquid in the well remains within a predetermined focal length range to thereby provide a substantially constant flowrateut of nebulised liquid. The nebuliser may also include a deflector baffle or fountain diverter (16) which acts to deflect the nebulised liquid fountain rising from the well. In order to reduce large droplets leaving the nebulisation chamber a circuitous or labyrinthine path is also provided between the well and the exit (13) of the nebulisation chamber

### FIELD OF THE INVENTION

The present invention relates to a method and apparatus for generating an aerosol. More particularly, the present invention relates to a nebuliser.

The invention has been developed primarily for use as an ultrasonic nebuliser and will be described hereinafter with reference to this application. However, it will be appreciated that the invention is not limited to this particular field of use.

### BACKGROUND OF THE INVENTION

Any discussion of the prior art throughout the specification should in no way be considered as an admission that such prior art is widely known or forms part of the common general knowledge in the field.

Nebulisers are widely employed in a number of applications, e.g. nebulisation of liquid fuel, moisturisation of air and for sterilisation purposes. One common application is in the medical field. Medical nebulisers provide an aerosol of medication for pulmonary delivery of drugs for the treatment of certain conditions and diseases. Nebulisers have applications for conscious, spontaneously-breathing patients and for controlled, ventilated patients.

There are a number of techniques that can be used to generate an aerosol. For example, in some nebulisers, a gas and a fluid are mixed together and directed against a baffle or diverter to cause nebulisation, such as disclosed in EP 0 191 018, WO 95/20411 and WO 95/25556 and U.S. Pat. No. 6,223,745. In other nebulisers, a quickly moving gas is moved over a fluid orifice. The negative pressure created by the flow of pressurised gas is a factor that contributes to drawing fluid out of the orifice and nebulising it. However, these nebulisers produce high noise when actuated. Other types of nebulisers utilise an energy source such as ultrasonic energy for directly producing an aerosol of liquid, such as disclosed in U.S. Pat. No. 6,152,383 and U.S. Pat. No. 6,283,118.

An important in consideration in nebuliser design is the timing and dosage regulation of the aerosolised fluid. In certain nebulisers, the fluid will be constantly aerosolised until the reservoir is depleted. This necessarily wastes the fluid during the patients exhaling cycle, is energy inefficient, means that a significant amount of drug needs to be charged to the device, and that only a single dose may be delivered per charge. Other designs include the provision of a manual trigger for the patient to start atomisation as they inhale. However, this necessarily requires skill on the part of the patient who must coordinate inhalation with the trigger operation.

Nebulisers that are intermittent and timed to nebulise upon detection of the patient's inhalation cycle are known. Intermittent nebulisation may adversely affect particle size and density of the formed aerosol. Also, these devices are typically complex in construction. One particular example of a nebuliser having inhalation-detection is disclosed in U.S. Pat. No. 6,116,233, where a sensor in the form of a microphone detects turbulent air flow during inhalation and causes the nebuliser to generate aerosol only during the inhalation phase of the breathing cycle. This device, however, only operates when the inhalation is sufficient to cause turbulent flow. Still further the inlet path for the air is complex passing various baffles and valves thereby interfering with smooth passage of the air into the nebulisation chamber.

Other important considerations include the particle size and uniformity of particles of the formed aerosol. As a general rule, the smaller the particles the better the penetration of the particles into the lungs and bronchial passageways. In particular, aerosols larger then 5 micron poorly penetrate the upper respiratory tract whereas those in the 0.2 to 2 micron range tend to have their maximum disposition in the lung parenchyma. Because of their mass and inertia, droplets substantially larger than about 5 micron which are inhaled by a patient will tend to collide with and collect on the walls of the respiratory tract before penetrating deep into the lungs. As the medicament must penetrate deep into the lungs to produce the desired therapeutic effect, medicament that never reaches the effective areas of the lungs is wasted and consequently increases the cost of the treatment.

Another important consideration relates to the control over the delivered dosage. Nebulisers previously available on the market have generally been designed to be used with drugs which have a wide therapeutic dose range, i.e. it has been possible to allow the dosage to be varied within wide limits without serious consequences, e.g. traditional asthma medication. In these instances the demand for exact and reproducible dosage has not been so stringent. Accordingly, the demands on the devices themselves have not been so stringent. However, the advent and availability of a number of powerful and usually very costly drugs which require a strictly controlled dosage regimen has imposed stringent demands on the dosing equipment. For example, control over the flow rate of aerosolised drug to the patient is important to ensure that a consistent and reproducible dose is delivered each time.

Ultrasonic nebulisers typically include a nebulisation chamber having a reservoir of liquid to be nebulised, an energy source in the form of an ultrasonic transducer to effect nebulisation and a delivery tube. The energy source and the reservoir are positioned adjacent each other and a contact medium provides energy transmission between the source and the liquid. Ultrasonic nebulisers may also include a fan to transport the nebulised aerosol to the patient. The configuration of these nebulisers is such that a large proportion of non-nebulised liquid (i.e. droplets) are returned to the well of liquid being nebulised, and in particular to the area of the active part of the fountain (i.e. the base). This results in reduced effectiveness and stability of the nebulisation process. These disadvantages were addressed in U.S. Pat. No. 3,901,443 where the ultrasonic transducer was placed at an angle to the surface of the nebulised liquid. In U.S. Pat. No. 4,410,139 a slotted partition was also employed such that the non-nebulised liquid tended to fall to the outside of a partition and so interfered less with the base. There are a number of disadvantages to these devices including: complexity, the need for a fan to effect aerosol transport, reduction of nebulisation efficiency due to the asymmetry of the transducer and the need to use a partition which affects the energy delivered to the liquid.

Several of these disadvantages were avoided in WO 94/08727. In this application, the nebulisation chamber is separated into 2 parts: a lower chamber containing the liquid reservoir to be nebulised, and an upper expansion chamber having an outlet tube for transport of the aerosol to the user. The chambers are divided by a partition having a central aperture for the nebulised fountain and peripheral apertures to promote the return of condensed non-nebulised droplets to the reservoir. However, despite the partition, droplets may still be returned to the base of the fountain or even transported to and inhaled by the patient. This may be due to the symmetry of the device.

Very few attempts have been made at controlling the flow rate of nebulised liquid emanating from such ultrasonic nebulisers. This may be due to the relative complexity of such an undertaking. For example, the flow rate may be a function of many factors including: volume of liquid initially charged to the device, the rate of liquid consumption, design of the transducer (shape and power, which affect the energy delivered to the liquid and in turn the nebulisation efficiency), the internal layout of the nebulisation chamber and potentially the type of drug being nebulised.

It is an object of the present invention to overcome or ameliorate at least one of the disadvantages of the abovementioned prior art, or to provide a useful alternative.

### DISCLOSURE OF THE INVENTION

According to a first aspect the present invention provides a nebuliser including a nebulisation chamber having a well adapted to contain a liquid to be nebulised, an energy source operatively associated with said well to nebulise said liquid, said energy source having a curved energy transmission surface thereby defining a focal point and focal length of energy produced by said source: and wherein said energy source is spaced from said well such that the distance between said focal point and said energy source intrudes into said well not greater than 50% of the focal length.

According to a second aspect the present invention provides a method of nebulising liquid comprising:
containing the liquid to be nebulised in a well;
providing an energy source having a curved energy transmission surface defining a focal point and focal length of energy produced by said source;
transmitting energy from said source to said well to thereby nebulise said liquid contained therein;
wherein said energy source is spaced from said well such that the distance between said focal point and said energy source intrudes into said well not greater than 50% of said focal length.

In a first embodiment, the energy source is an ultrasonic transducer preferably constructed of piezoelectric ceramic material and having a parabolic energy transmission surface. The well containing the liquid to be nebulised can be operatively associated with the energy source by any suitable mechanism however a contact medium between the well and energy source is preferred. The contact medium preferably has a high energy transmission efficiency and can be selected from the group consisting of water, rubbery polymer, gel, oil etc.

As discussed above the well is spaced from the energy source such that the distance between the focal point and the energy source intrudes into the well by no greater than 50% of the focal length. In further preferred embodiments, this intrusion is no greater than 40%, preferably no greater than 30%, more preferably no greater than 20% and most preferably no greater than 10%.

In conjunction with the preferred well construction which is relatively shallow and/or adapted to contain a relatively thin/shallow layer of fluid the present invention has significant advantages over the prior art.

In other embodiments, the present invention allows the well to be positioned such that the focal point is positioned beneath the surface and within the volume of the nebulisable liquid thereby gaining maximum efficiency from the energy source. Accordingly only a small well with the precise quantity of liquid to be nebulised is required. This is clearly advantageous compared with the prior art which generally comprise large wells containing significant “oversupply” of liquid since the well extends from a position either directly on or adjacent to the energy transmission surface to the focal point of the energy source. However, in alternative embodiments the spacing of the well from the energy source is such that the focal point is positioned above the surface of the nebulisable liquid. In one embodiment, the well contains up to 3 mL of fluid, preferably up to 2 mL and more preferably up to 1 mL of liquid to be nebulised. While not limited to this application, the inventive apparatus and process have been shown to be particularly suitable for use where the liquid is a drug or a solution/suspension of a drug. Once again this has clear and significant advantages over the prior art since it allows precise dosages of such drugs to be contained within the well and released during nebulisation.

The well can be constructed from many suitable materials but is preferably produced from a high performance thermoplastic material such as PEEK.

According to a third aspect the present invention provides a nebuliser including a nebulisation chamber having a well adapted to contain a liquid to be nebulised, an energy source operatively associated with said well to nebulise said liquid and thereby produce a fountain of liquid rising from said well, and a deflector baffle positioned directly above said well and adapted to deflect said liquid fountain rising from said well.

In a preferred embodiment the deflector baffle is positioned to deflect substantially all the liquid that impinges upon the deflector baffle in a direction away from the axis of the liquid fountain.

In another preferred embodiment, the liquid fountain is deflected substantially to one side of its axis.

In yet another embodiment, the deflector baffle is placed intermediate the well and the unhindered apex of the liquid fountain. As it will be understood by persons skilled in the art, the term “unhindered apex” of the liquid fountain refers to the height or apex of the liquid fountain as generated by the energy source with no redirection or deflection by the deflector baffle.

As is known by persons skilled in the art, upon actuation of the energy source, such as an ultrasonic transducer, a fountain of liquid is formed in the well and rises from the well to thereby nebulise the liquid. In many cases this fountain is left unhindered to rise to its maximum height. Such an arrangement, however, can be inefficient since, at its apex, any liquid which is not nebulised will fall back down along the apex of the fountain to thereby decrease the energy of the fountain. In many cases the fountain falling on itself substantially increases the energy requirement of the ultrasonic transducer.

In accordance with the present invention, the fountain of nebulised liquid is deflected preferably prior to its unhindered apex, to avoid the fountain fall on itself and thereby reducing its energy.

In another preferred embodiment, the deflector baffle is adapted to deflect the liquid which impinges on it, to at least one side of the fountain axis. It is also preferred that this deflected liquid is recycled to the well for further nebulisation.

As discussed above it is most preferred that the deflector baffle is positioned intermediate the well and the unhindered apex of the fountain of liquid.

In other embodiments the deflector baffle is shaped as an inverted U wherein the apex of the inverted U is spaced from the axis of the fountain. The U-shaped deflector baffle may also include a deflection surface adjacent its apex, wherein the fountain impinges directly on the deflection surface during nebulisation.

According to a fourth aspect the present invention provides a method of nebulising liquid in a nebuliser, the nebuliser having a nebulisation chamber with a well adapted to contain a liquid to be nebulised, an energy source operatively associated with the well to nebulise the liquid, the method comprising providing a deflector baffle directly above the well and deflecting liquid rising from the well upon nebulisation of the liquid.

According to a fifth aspect the present invention provides a nebuliser including:
a nebulisation chamber having a well adapted to contain a liquid to be nebulised and an exit to allow egress of the nebulised liquid; and
an energy source operatively associated with the well for nebulising the liquid wherein;
the nebulisation chamber defines a circuitous path between the well and the exit.

In yet a further embodiment, the circuitous path is defined by one or more baffles within the nebulisation chamber. Preferably the baffles are numbered and positioned such that any liquid entrained in a fountain of nebulised liquid is substantially returned to the well for re-nebulisation, and the nebulised liquid is free to exit the chamber.

Preferably actuation of the energy source produces a fountain of nebulised liquid, and the nebulised liquid has a particle size below a predetermined particle size. Desirably the predetermined particle size is 5 micron. Preferably the predetermined particle size is 1 micron. The nebulised liquid having a particle size below the predetermined particle size is substantially neutrally buoyant. Preferably the baffles are numbered and positioned such that any nebulised liquid below the predetermined particle size is free to exit the chamber, and any liquid above the predetermined particle size is caught on the baffles and returned to the well for re-nebulisation.

According to a sixth aspect the present invention provides a method of nebulising a liquid comprising:
containing the liquid to be nebulised in a well, positioning the well in a nebulisation chamber having an exit to allow gress of a nebulised liquid, and
defining a circuitous path between the well and the exit.

The present applicants have found that unlike the prior art certain advantages arise in providing a circuitous path between the well and the exit of the nebulisation chamber. In most of the prior art, there is a direct line between the well and the exit such that the nebulised liquid can proceed unhindered from the fountain of liquid formed by nebulisation to the exit. The Applicants have taken an entirely different approach.

The Applicants have found that by providing a circuitous path, this reduces the possibility of large non-nebulised droplets of liquid exiting the nebuliser but does not unnecessarily impede passage of nebulised liquid through the nebulisation chamber to exit the device.

The nebulised liquid droplets smaller than a particular predetermined size effectively have a “neutral buoyancy”. In other words they simply float within the nebulisation chamber. Accordingly, the circuitous path does not significantly impact on the passage of such nebulised liquid or aerosol through the device. Providing a circuitous or labyrinthine passageway, however, reduces the possibility of large droplets leaving the device. Such large droplets are not only ineffective in terms of drug delivery but due to the highly efficient low dosage arrangement of the present invention, they can significantly impact on the quantity of liquid remaining in the well and thereby negatively impact on subsequent dosages.

By suitable arrangement of the baffles, such large droplets can be “caught” by impacting on the baffle for subsequent return/recycle to the well.

According to a seventh aspect the present invention provides a nebuliser comprising nebulisation chamber having a well adapted to contain a nebulisable liquid and an energy source spaced from and operatively associated with said well to nebulise said liquid, said energy source including a curved energy transmission surface thereby defining an energy focal point and a focal length between said energy source and said focal point, wherein said well is shaped such that during nebulisation the level of nebulisable liquid remains within a predetermined focal length range to thereby provide a substantially constant flow rate of nebulised liquid.

Preferably the predetermined focal length range is such that flow rate remains within 10% of maximum flow rate, most preferably within 5% of maximum flow rate.

The maximum flow rate may be up to 1.5 mL/min, preferably 1.2 mL/min, more preferably 1.0 mL/min and most preferably 0.8 mL/min. However, the maximum flow rate may be 0.6 mL/min or 0.4 mL/min. In preferred embodiments the maximum flow rate is between 0.8 and 1.2 mL/min. More preferably the maximum flow rate is between 0.9 and 1.0 mL/min.

The applicants have found that the nebulisation efficiency and the flow rate of nebulised liquid is a strong function of the positioning of the surface of the nebulisable liquid relative to the focal point and energy source.

In particular it has been found that there is a predetermined range along the focal length where upon activation of the energy source, the flow rate of the nebulised liquid is at a consistent or at least substantially consistent level. This is important for a number of reasons, including that it provides a consistent level of drug delivery to the user.

The applicants have found that, as will be discussed below, a maximum flow rate of nebulised liquid is obtained where the focal point is just beneath the surface of the nebulisable liquid. However the flow rate reduces sharply as the level in the liquid drops such that the focal point is positioned slightly above the liquid surface. At a point where the focal point is above the liquid surface, the applicants have found that the nebulised liquid flow rate may be supplied at a consistent rate for a given energy transmission up until a second point approaching the well being dry where, of course, the flow rate of the nebulised liquid drops to zero.

By appropriate design of the well and spacing of the energy source from the well the level of nebulisable liquid in the well can be maintained in the predetermined range to provide a consistent flow of the nebulised liquid drug to the user. Preferably the predetermined focal length range providing the substantially constant flow rate of nebulised liquid corresponds to a volume contained in the well of between 1 and 6 mL. In other embodiments the predetermined focal length range providing the substantially constant flow rate of nebulised liquid corresponds to a volume contained in the well of between 2 and 4 mL. In yet further embodiments the predetermined focal length range providing the substantially constant flow rate of nebulised liquid corresponds to a volume contained in the well of between 2 and 3 mL.

Preferably the well is designed with a wide base such that the liquid is relatively shallow compared with the prior art devices. This assists in reducing the change in depth of the liquid drug during nebulisation, thereby keeping the liquid within the predetermined focal length range for consistent flow rate.

It is also preferable that the bottom wall of the well is either flat or preferably slightly tapered downwardly. Most preferably the bottom wall of the well contains a frusto-conical section disposed about a base portion at its lowest most point thereby forming a “waste reservoir”. In other words, the preferred well is in the form of a “funnel”, having a lower portion in the form of a shallow well and being connected to an upper portion having at least one tapered wall. In use this reservoir is positioned at the lowest point of the well. The liquid in this well generally falls below the predetermined focal length range for consistent flow rate. Accordingly when the level of liquid in the drug well reaches the waste reservoir the nebuliser no longer provides a constant flow rate of nebulised liquid and accordingly this material can be considered waste.

As discussed above, the energy source has a curved energy transmission surface thereby defining a focal point and focal length of energy produced by the source. The energy source is preferably spaced from the well such that the focal point is positioned above the surface of the nebulisable liquid, and in preferred embodiments the distance between the focal point and the surface of the nebulisable liquid is not greater than 50% of the focal length. In further preferred embodiments, this distance is not greater than 40%, preferably not greater than 30%, more preferably not greater than 20% and most preferably not greater than 10%. Preferably the energy source is disposed directly beneath the well and the focal point is positioned above the surface of the nebulisable liquid when the nebuliser is held in a substantially upright position.

In a related embodiment, the well contains up to 8 mL of nebulisable liquid, preferably up to 6 mL, more preferably up to 5 mL, and most preferably up to 4 mL. Whilst not limited to this application, the inventive apparatus and process have been shown to be particularly suitable for use where the liquid is a drug or a solution/suspension of a drug. Once again this has clear and significant advantages over the prior art since it allows precise dosages of such drugs to be contained within the well and released during nebulisation.

The present applicants have found that the drug volume and drug wastage can be minimised, and a reproducible and consistent nebulised drug flow rate can be provided by the combination of shaping of the drug well such that the change in surface height of the remaining liquid remains in the predetermined focal length range.

Furthermore, the applicants have surprisingly found that the time to nebulisation, i.e. the time between actuation of the energy source to the time at which a constant flow rate is achieved, is similar if the energy focal point is positioned above the surface of the remaining liquid for a range of heights.

According to an eighth aspect the present invention provides a method of nebulising a nebulisable liquid comprising:
providing a nebulisation chamber having a well adapted to contain a nebulisable liquid;
providing an energy source spaced from and operatively associated with said well to nebulise said liquid, said energy source including a curved energy transmission surface thereby defining an energy focal point and a focal length between said energy source and said focal point; and
nebulising said nebulisable liquid,
wherein said well is shaped such that during nebulisation the level of liquid remains within a predetermined focal length range to thereby provide a substantially constant flow rate of nebulised liquid.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”.

Other than in the operating examples, or where otherwise indicated, all numbers expressing quantities of ingredients or reaction conditions used herein are to be understood as modified in all instances by the term “about”.

### DEFINITIONS

In describing the present invention, the following terminology will be used in accordance with the definitions set out below.

“Aerosol” refers to liquid particles suspended in a gas with particle sizes about 0.1 to 10 microns in diameter. Aerosols are typically charged and have substantially neutral buoyancy. “Mist” refers to liquid droplets suspended in a gas with particle sizes about 40 to 500 microns in diameter.

“Drug” means any substance that is used in the prevention, diagnosis, alleviation, treatment or cure of a condition. The terms “drug”, “compound”, “medication”, “active agent” and “pharmacologically active agent” are used herein interchangeably.

“Drug composition” refers to a composition that comprises only pure drug, two or more drugs in combination, or one or more drugs in combination with additional components. Additional components can include, for example, pharmaceutically acceptable excipients, carriers, solvents, and surfactants.

By “pharmaceutically acceptable,” such as in the recitation of a “pharmaceutically acceptable carrier,” or a “pharmaceutically acceptable acid addition salt,” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. “Pharmacologically active” (or simply “active”) as in a “pharmacologically active” derivative or metabolite, refers to a derivative or metabolite having the same type of pharmacological activity as the parent compound and approximately equivalent in degree. When the term “pharmaceutically acceptable” is used to refer to a derivative (e.g., a salt) of an active agent, it is to be understood that the compound is pharmacologically active as well. “Carriers” or “vehicles” as used herein refer to conventional pharmaceutically acceptable carrier materials suitable for drug administration, and include any such materials known in the art that are nontoxic and do not interact with other components of a pharmaceutical composition or drug delivery system in a deleterious manner. For example, the drug may be in solution or in suspension.

The terms “treating” and “treatment” as used herein refer to the ability to effect a response relative to that individual's response in the absence of pharmacotherapy as provided herein.

By an “effective” amount or a “therapeutically effective amount” of a drug or pharmacologically active agent is meant a nontoxic but sufficient amount of the drug or agent to provide the desired effect. The amount that is “effective,” however, will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation.

By “as-needed” dosing, also referred to as “pro re nata” dosing, “pm” dosing, and “on-demand” dosing or administration, is meant the administration of an active agent at a time just prior to the time at which drug efficacy is wanted and within a time interval sufficient to provide for the desired therapeutic effect. “As-needed” administration herein does not involve priming doses or chronic administration, “chronic” meaning administration at regular time intervals on an ongoing basis.

### Active Agents

Before describing the present invention in detail, it is to be understood that this invention is not limited to specific active agents, dosing regimens, or the like, as such may vary. It is also to be understood that the terminology used herein is for the purpose of describing particular embodiments only, and is not intended to be limiting.

Any suitable drug compound may be used with the device of the invention. Drugs that can be used include, for example but not limitation, drugs of one of the following classes: anaesthetics, anticonvulsants, antidepressants, antidiabetic agents, antidotes, antiemetics, antihistamines, anti-infective agents, antineoplastics, antiparkisonian drugs, antirheumatic agents, antipsychotics, anxiolytics, appetite stimulants and suppressants, blood modifiers, cardiovascular agents, central nervous system stimulants, drugs for Alzheimer's disease management, drugs for cystic fibrosis management, diagnostics, dietary supplements, drugs for sexual dysfunction in men and women, gastrointestinal agents, hormones, drugs for the treatment of alcoholism, drugs for the treatment of addiction, immunosuppressives, mast cell stabilizers, migraine preparations, motion sickness products, drugs for multiple sclerosis management, muscle relaxants, nonsteroidal anti-inflammatories, opioids, other analgesics and stimulants, ophthalmic preparations, osteoporosis preparations, prostaglandins, respiratory agents, sedatives and hypnotics, skin and mucous membrane agents, smoking cessation aids, Tourette's syndrome agents, urinary tract agents, and vertigo agents.

Typically, where the drug is an anaesthetic, it is selected from one of the following compounds: ketamine and lidocaine.

Typically, where the drug is an anticonvulsant, it is selected from one of the following classes: GABA analogs, tiagabine, vigabatrin; barbiturates such as pentobarbital; benzodiazepines such as clonazepam; hydantoins such as phenytoin; phenyltriazines such as lamotrigine; miscellaneous anticonvulsants such as carbamazepine, topiramate, valproic acid, and zonisamide.

Typically, where the drug is an antidepressant, it is selected from one of the following categories:
1) tricyclic antidepressants (TCADs or TCAs), such as clomipramine, imipramine, lofepramine, nortriptyline, amitriptyline, desipramine, dosulepin, doxepin, trimipramine, amoxapine, trazodone, amineptine, dothiepin, iprindole, opipramol, propizepine, protriptyline, quinupramine and fluphenazine;
2) selective serotonin and noradrenaline reuptake inhibitors (SNRIs), such as venlafaxine and milnacipran;
3) selective serotonin reuptake inhibitors (SSRIs), such as citalopram, escitalopram, fluoxetine, fluvoxamine, paroxetine, clovoxamine, femoxetine, ifoxetine, viqualine, zimeldine and sertraline;
4) selective noradrenaline reuptake inhibitors (NARIs), such as reboxetine, desipramine, oxaprotiline and melitracen;
5) noradrenaline and selective serotonin antidepressants (NASSAs), such assibutramine andmirtazapine;
6) monoamine oxidase inhibitors (MAOIs), such asmoclobemide, tranylcypromine, brofaromine, clorgyline, isocarboxazid, nialamide, pirlindole, selegiline, toloxatone, viloxazine and phenelzine;
7) lithium salts, such as lithium carbonate and lithium citrate;
8) GABA potentiators, such asvalproic acid;
9) thioxanthenes, such asflupentixol;
10) tetracyclic antidepressants, such as maprotiline, levoprotiline, mianserin; and
11) further agents which may not fit into the above mentioned categories, such as bupropion, carbamazepine, tryptophan, amesergide, benactyzine, butriptyline, cianopramine, demexiptiline, dibenzepin, dimetacrine, etoperidone, fezolamine, medifoxamine, metapramine, methylphenidate, minaprine, nomifensine, oxaflozane, oxitriptan, rolipram, setiptiline, teniloxazine, tianeptine, tofenacin and nefazodone.

The term antidepressants, as used herein, may also encompass antipsychotic drugs which may also be used in the compositions of the present invention. Such antipsychotic drugs include, for example, aripiprazole, chlorpromazine, zuclopenthixol, clozapine, flupentixol, sulpiride, perphenazine, fluphenazine, haloperidol, thioridazine, pericyazine, levomeptomazine, pimozide, oxypertine, pipotiazine, promazine, risperidone, quetiapine, amisulpride, trifluoperazine, prochlorperazine, zotepine and olanzapine.

Typically, where the drug is an antidiabetic agent, it is selected from one of the following compounds: pioglitazone, rosiglitazone, and troglitazone.

Typically, where the drug is an antidote, it is selected from one of the following compounds: edrophonium chloride, flumazenil, deferoxamine, nalmefene, naloxone, and naltrexone.

Typically, where the drug is an antiemetic, it is selected from one of the following compounds: alizapride, azasetron, benzquinamide, bromopride, buclizine, chlorpromazine, cinnarizine, clebopride, cyclizine, diphenhydramine, diphenidol, dolasetron, droperidol, granisetron, hyoscine, lorazepam, dronabinol, metoclopramide, metopimazine, ondansetron, perphenazine, promethazine, prochlorperazine, scopolamine, triethylperazine, trifluoperazine, triflupromazine, trimethobenzamide, tropisetron, domperidone, and palonosetron.

Typically, where the drug is an antihistamine, it is selected from one of the following compounds: astemizole, azatadine, brompheniramine, carbinoxamine, cetrizine, chlorpheniramine, cinnarizine, clemastine, cyproheptadine, dexmedetomidine, diphenhydramine, doxylamine, fexofenadine, hydroxyzine, loratidine, promethazine, pyrilamine and terfenidine.

Typically, where the drug is an anti-infective agent, it is selected from one of the following classes: antivirals such as efavirenz; AIDS adjunct agents such as dapsone; aminoglycosides such as tobramycin; antifungals such as fluconazole; antimalarial agents such as quinine; antituberculosis agents such as ethambutol; P-lactams such as cefinetazole, cefazolin, cephalexin, cefoperazone, cefoxitin, cephacetrile, cephaloglycin, cephaloridine; cephalosporins, such as cephalosporin C, cephalothin; cephamycins such as cephamycin A, cephamycin B, and cephamycin C, cephapirin, cephradine; leprostatics such as clofazimine; penicillins such as ampicillin, amoxicillin, hetacillin, carfecillin, carindacillin, carbenicillin, amylpenicillin, azidocillin, benzylpenicillin, clometocillin, cloxacillin, cyclacillin, methicillin, nafcillin, 2-pentenylpenicillin, penicillin N, penicillin O, penicillin S, penicillin V, dicloxacillin; diphenicillin; heptylpenicillin; and metampicillin; quinolones such as ciprofloxacin, clinafloxacin, difloxacin, grepafloxacin, norfloxacin, ofloxacine, temafloxacin; tetracyclines such as doxycycline and oxytetracycline; miscellaneous anti-infectives such as linezolide, trimethoprim and sulfamethoxazole.

Typically, where the drug is an anti-neoplastic agent, it is selected from one of the following compounds: droloxifene, tamoxifen, and toremifene.

Typically, where the drug is an antiparkisonian drug, it is selected from one of the following compounds: anantadine, baclofen, biperiden, benztropine, orphenadrine, procyclidine, trihexyphenidyl, levodopa, carbidopa, andropinirole, apomorphine, benserazide, bromocriptine, budipine, cabergoline, eliprodil, eptastigmine, ergoline, galanthamine, lazabemide, lisuride, mazindol, memantine, mofegiline, pergolide, piribedil, pramipexole, propentofylline, rasagiline, remacemide, ropinerole, selegiline, spheramine, terguride, entacapone, and tolcapone.

Typically, where the drug is an antirheumatic agent, it is selected from one of the following compounds: diclofenac, hydroxychloroquine and methotrexate.

Typically, where the drug is an antipsychotic, it is selected from one of the following compounds: acetophenazine, alizapride, amisulpride, amoxapine, amperozide, aripiprazole, benperidol, benzquinamide, bromperidol, buramate, butaclamol, butaperazine, carphenazine, carpipramine, chlorpromazine, chlorprothixene, clocapramine, clomacran, clopenthixol, clospirazine, clothiapine, clozapine, cyamemazine, droperidol, flupenthixol, fluphenazine, fluspirilene, haloperidol, loxapine, melperone, mesoridazine, metofenazate, molindrone, olanzapine, penfluridol, pericyazine, perphenazine, pimozide, pipamerone, piperacetazine, pipotiazine, prochliorperazine, promazine, quetiapine, remoxipride, risperidone, sertindole, spiperone, sulpiride, thioridazine, thiothixene, trifluperidol, triflupromazine, trifluoperazine, ziprasidone, zotepine, and zuclopenthixol.

Typically, where the drug is an anxiolytic, it is selected from one of the following compounds: alprazolam, bromazepam, oxazepam, buspirone, hydroxyzine, mecloqualone, medetomidine, metomidate, adinazolam, chlordiazepoxide, clobenzepam, flurazepam, lorazepam, loprazolam, midazolam, alpidem, alseroxlon, amphenidone, azacyclonol, bromisovalum, captodiamine, capuride, carbcloral, carbromal, chloral betaine, enciprazine, flesinoxan, ipsapiraone, lesopitron, loxapine, methaqualone, methprylon, propanolol, tandospirone, trazadone, zopiclone, and zolpidem.

Typically, where the drug is an appetite stimulant, it is dronabinol.

Typically, where the drug is an appetite suppressant, it is selected from one of the following compounds: fenfluramine, phentermine and sibutramine.

Typically, where the drug is a blood modifier, it is selected from one of the following compounds: cilostazol and dipyridamol.

Typically, where the drug is a cardiovascular agent, it is selected from one of the following compounds: benazepril, captopril, enalapril, quinapril, ramipril, doxazosin, prazosin, clonidine, labetolol, candesartan, irbesartan, losartan, telmisartan, valsartan, disopyramide, flecanide, mexiletine, procainamide, propafenone, quinidine, tocainide, amiodarone, dofetilide, ibutilide, adenosine, gemfibrozil, lovastatin, acebutalol, atenolol, bisoprolol, esmolol, metoprolol, nadolol, pindolol, propranolol, sotalol, diltiazem, nifedipine, verapamil, spironolactone, bumetanide, ethacrynic acid, furosemide, torsemide, amiloride, triamterene, and metolazone.

Typically, where the drug is a central nervous system stimulant, it is selected from one of the following compounds: amphetamine, brucine, caffeine, dexfenflurainine, dextroamphetamine, ephedrine, fenfluramine, mazindol, methyphenidate, pemoline, phentermine, sibutramine, and modafinil.

Typically, where the drug is a drug for Alzheimer's disease management, it is selected from one of the following compounds: donepezil, galanthamine and tacrin.

Typically, where the drug is a drug for cystic fibrosis management, it is selected from one of the following compounds: CPX, IBMX, XAC and analogues; 4-phenylbutyric acid; genistein and analogous isoflavones; and milrinone.

Typically, where the drug is a diagnostic agent, it is selected from one of the following compounds: adenosine and aminohippuric acid.

Typically, where the drug is a dietary supplement, it is selected from one of the following compounds: melatonin and vitamin-E.

Typically, where the drug is a drug for sexual dysfunction in men and women, it is selected from one of the following compounds: tadalafil, sildenafil, vardenafil, apomorphine, apomorphine diacetate, phentolamine, chlorpromazine, chlomipramine, prostaglandin, yohimbine, melanocortin, vasoactive intestinal polypeptide (vip) and papaverine.

Typically, where the drug is a gastrointestinal agent, it is selected from one of the following compounds: loperamide, atropine, hyoscyamine, famotidine, lansoprazole, omeprazole, and rebeprazole.

Typically, where the drug is a hormone, it is selected from one of the following compounds: testosterone, estrogen, progesterone, cortico steroids.

Typically, where the drug is a drug for the treatment of alcoholism, it is selected from one of the following compounds: naloxone, naltrexone, and disulfiram.

Typically, where the drug is a drug for the treatment of addiction it is buprenorphine.

Typically, where the drug is an immunosupressive, it is selected from one of the following compounds: mycophenolic acid, cyclosporin, azathioprine, tacrolimus, and rapamycin.

Typically, where the drug is a mast cell stabilizer, it is selected from one of the following compounds: cromolyn, pemirolast, and nedocromil.

Typically, where the drug is a drug for migraine headache, it is selected from one of the following compounds: almotriptan, alperopride, codeine, dihydroergotamine, ergotamine, eletriptan, frovatriptan, isometheptene, lidocaine, lisuride, metoclopramide, naratriptan, oxycodone, propoxyphene, rizatriptan, sumatriptan, tolfenamic acid, zolmitriptan, amitriptyline, atenolol, clonidine, cyproheptadine, diltiazem, doxepin, fluoxetine, lisinopril, methysergide, metoprolol, nadolol, nortriptyline, paroxetine, pizotifen, pizotyline, propanolol, protriptyline, sertraline, timolol, and verapamil.

Typically, where the drug is a motion sickness product, it is selected from one of the following compounds: diphenhydramine, promethazine, and scopolamine.

Typically, where the drug is a drug for multiple sclerosis management, it is selected from one of the following compounds: bencyclane, methylprednisolone, mitoxantrone, and prednisolone.

Typically, where the drug is a muscle relaxant, it is selected from one of the following compounds: baclofen, chlorzoxazone, cyclobenzaprine, methocarbamol, orphenadrine, quinine, and tizanidine.

Typically, where the drug is a nonsteroidal anti-inflammatory, it is selected from one of the following compounds: aceclofenac, acetaminophen, alminoprofen, amfenac, aminopropylon, amixetrine, aspirin, benoxaprofen, bromfenac, bufexamac, carprofen, celecoxib, choline, salicylate, cinchophen, cinmetacin, clopriac, clometacin, diclofenac, diflunisal, etodolac, fenoprofen, flurbiprofen, ibuprofen, indomethacin, indoprofen, ketoprofen, ketorolac, mazipredone, meclofenamate, nabumetone, naproxen, parecoxib, piroxicam, pirprofen, rofecoxib, sulindac, tolfenamate, tolmetin, and valdecoxib.

Typically, where the drug is an opioid, it is selected from one of the following compounds: alfentanil, allylprodine, alphaprodine, anileridine, benzylmorphine, bezitramide, buprenorphine, butorphanol, carbiphene, cipramadol, clonitazene, codeine, dextromoramide, dextropropoxyphene, diamorphine, dihydrocodeine, diphenoxylate, dipipanone, fentanyl, hydromorphone, L-alpha acetyl methadol, lofentanil, levorphanol, meperidine, methadone, meptazinol, metopon, morphine, nalbuphine, nalorphine, oxycodone, papaveretum, pethidine, pentazocine, phenazocine, remifentanil, sufentanil, and tramadol.

Typically, where the drug is another analgesic it is selected from one of the following compounds: apazone, benzpiperylon, benzydramine, caffeine, clonixin, ethoheptazine, flupirtine, nefopam, orphenadrine, propacetamol, and propoxyphene.

Typically, where the drug is an opthalmic preparation, it is selected from one of the following compounds: ketotifen and betaxolol.

Typically, where the drug is an osteoporosis preparation, it is selected from one of the following compounds: alendronate, estradiol, estropitate, risedronate and raloxifene.

Typically, where the drug is a prostaglandin, it is selected from one of the following compounds: epoprostanol, dinoprostone, misoprostol, and alprostadil.

Typically, where the drug is a respiratory agent, it is selected from one of the following compounds: albuterol, ephedrine, epinephrine, fomoterol, metaproterenol, terbutaline, budesonide, ciclesonide, dexamethasone, flunisolide, fluticasone propionate, triancinolone acetonide, ipratropium bromide, pseudoephedrine, theophylline, montelukast, zafirlukast, ambrisentan, bosentan, enrasentan, sitaxsentan, tezosentan, iloprost, treprostinil, and pirfenidone

Typically, where the drug is a sedative and hypnotic, it is selected from one of the following compounds: butalbital, chlordiazepoxide, diazepam, estazolam, flunitrazepam, flurazepam, lorazepam, midazolam, temazepam, triazolam, zaleplon, zolpidem, and zopiclone.

Typically, where the drug is a skin and mucous membrane agent, it is selected from one of the following compounds: isotretinoin, bergapten and methoxsalen.

Typically, where the drug is a smoking cessation aid, it is selected from one of the following compounds: nicotine and varenicline.

Typically, where the drug is a Tourette's syndrome agent, it is pimozide.

Typically, where the drug is a urinary tract agent, it is selected from one of the following compounds: tolteridine, darifenicin, propantheline bromide, and oxybutynin.

Typically, where the drug is a vertigo agent, it is selected from one of the following compounds: betahistine and meclizine.

### BRIEF DESCRIPTION OF THE DRAWINGS

Preferred embodiments of the invention will now be described, by way of example only, with reference to the accompanying drawings in which:

FIG. 1 is a side cut-away view of a nebuliser according to a first embodiment of the present invention, shown in an inoperative position;

FIG. 2 is a view similar to FIG. 1 but showing the aerosol outlet tube rotated into an operative position;

FIG. 3 is a view similar to FIG. 2, but showing the nebuliser in operation and nebulised liquid being released;

FIG. 4 is a cut-away side view of a nebuliser according to a second embodiment of the present invention, shown prior to operation;

FIG. 5 is a view similar to FIG. 4 but showing the nebuliser in operation and nebulised liquid being released; and

FIG. 6 is a graph of nebulised fluid flow rate (F) versus the volume remaining in the drug well (V) for the nebuliser according to the second embodiment of the present invention.

### PREFERRED EMBODIMENT OF THE INVENTION

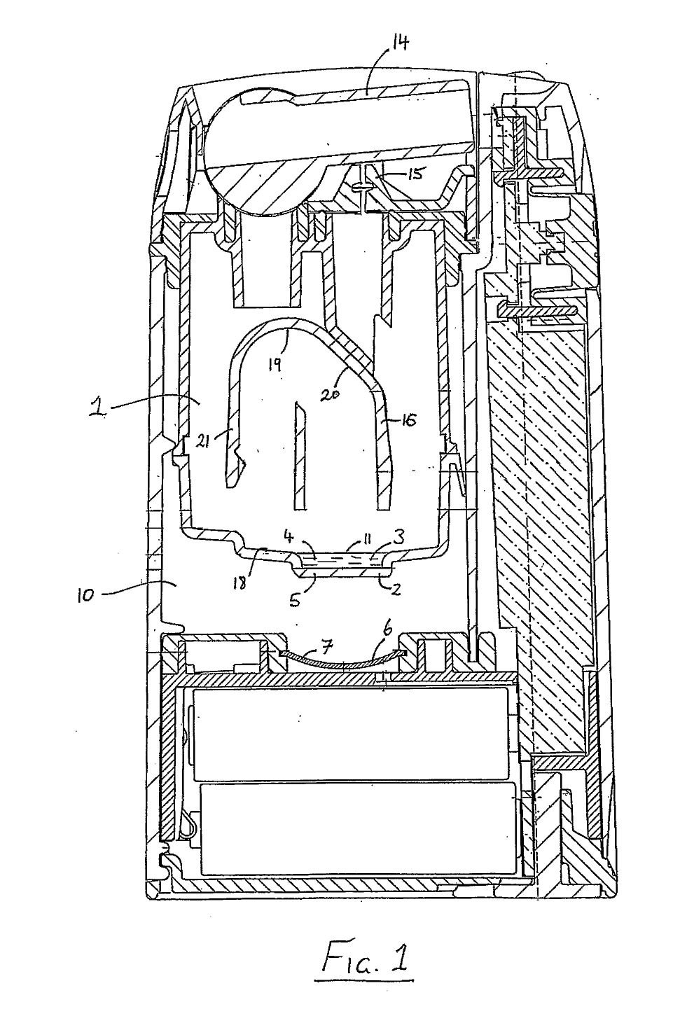

Referring initially to FIGS. 1-3, the nebuliser includes a nebulisation chamber 1 having a well 2 adapted to contain a liquid 3 to be nebulised. Preferably, the liquid 3 is a drug solution 4. It will be appreciated that the concentration of the liquid can be varied to suit the delivered dose. The well 2 is preferably disposed at the deepest part of the nebulisation chamber 1 and is shaped such that the drug solution 4 it contains is in the form of a relatively shallow pool. The well 2 may contain any amount of drug according to the size of the nebuliser. However, in the preferred embodiment the well contains up to 3 mL of liquid. The base 5 of the well is typically formed of a high performance thermoplastic material, eg polyetheretherketone (PEEK), and is sufficiently thin for lossless acoustic transmission.

An energy source in the form of an ultrasonic transducer 6 is operatively associated with the well 2 for nebulising the drug 4. The ultrasonic transducer is desirably made of a piezoelectric ceramic material and has a curved parabolic energy transmission surface 7 which defines a focal point 8 and a focal length 9. The ultrasonic transducer 6 is operatively associated with the well 2 preferably by way of a contact medium 10 which extends between the electronic transducer 6. The contact medium 10 preferably has a relatively high energy transmission efficiency and should desirably have similar acoustic properties to water i.e. wave velocity, acoustic impedance, etc. The contact medium 10 may be chosen from rubbery polymers, hydrogels, oils, etc, however the contact medium 10 is preferably water. The sterility of the contact medium 10 is not important as it does not enter the nebulisation chamber 1 or well 2. However, if non-sterilised water is used the water should be replaced at regular intervals.

The ultrasonic transducer 6 is spaced from the well 2 such that the distance between the focal point 8 and the parabolic surface 7 intrudes into the well 2 less than about 50% of the focal length 9. Preferably the focal length 9 intrudes into the well 2 less than 40%, more preferably less than 30%, even more preferably less than 20% and most preferably less than 10%. Such can be accomplished by either spacing of the well 2 and/or providing a relatively shallow well 2 as shown in the Figures. In one embodiment, the spacing is such that the focal point 8 is disposed just beneath the surface 11 of the drug solution 4 in the well 2. However, in other embodiments the spacing is such that the focal point 8 is disposed above the surface 11 of the drug solution 4 in the well 2.

Upon actuation of the ultrasonic transducer 6, ultrasonic energy is transmitted through the contact medium 10 and focussed into the well 2. The ultrasonic energy causes the liquid 3 to form an upwardly directed fountain 12 which rises from the well 2. It is understood that aerosol 13 escapes from the surface of the liquid including the surface of the fountain 12. The resultant aerosol 13 or nebulised liquid escapes into the nebulisation chamber 1 from where it is able to escape the device e.g. by inhalation through aerosol outlet 14.

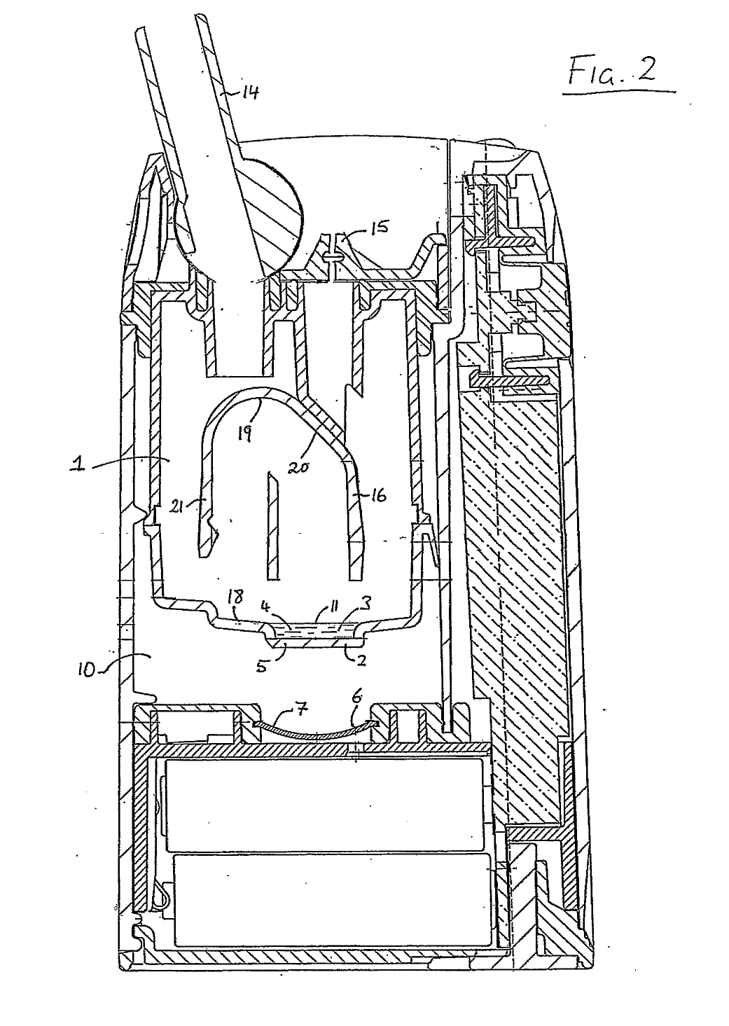

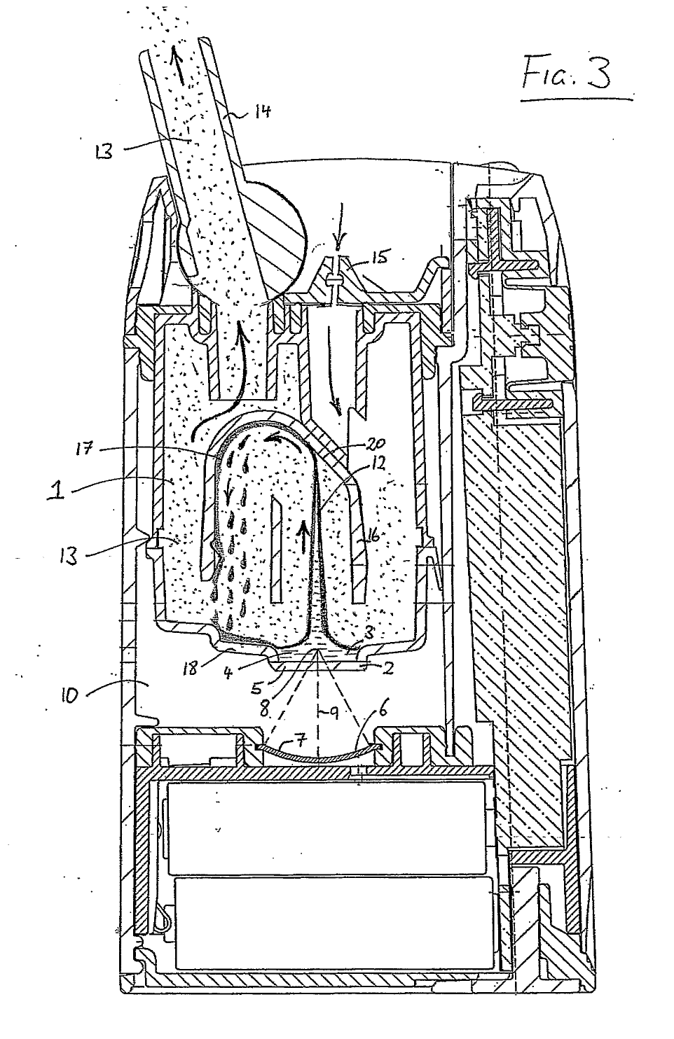

In use, the nebuliser is firstly charged with a liquid 3 to be nebulised via installation of well 2 which, in one embodiment, is replaceable as a cartridge, as shown in FIG. 1. The aerosol outlet 14 is then rotated into an operative position, as shown in FIG. 2, and the energy source is activated. The patient then inhales from the aerosol outlet 14 by mouth drawing air through the nebuliser from air inlet 15. The energy transmitted from the energy transmission surface 7 to the well 2 nebulises the liquid e.g. drug into an aerosol 13, as best shown in FIG. 3. The patient continues to inhale to receive the full dose of aerosolised drug 13. Once the dose is administered the aerosol outlet 14 is rotated back into an inoperative position, as best shown in FIG. 1, for storage thereby sealing the nebulisation chamber 1. The nebuliser is ready then for re-use as required by the patient.

As discussed above, in conjunction with the well construction as shown in FIGS. 1 to 3, which is relatively shallow and/or adapted to contain a relatively thin/shallow layer of fluid, the aforementioned design has significant advantages over the prior art including gaining maximum efficiency from the energy source 6, more efficient and better control of nebulisation by direct positioning of the well 2 at the focal point 8 of the energy source 6 etc.

The present invention is also suitable for a wide range of applications including but not limited to use in the treatment of sexual dysfunction in men and women such as erectile dysfunction etc. The nebuliser has excellent usage in this environment by means of its fast, effective and accurate dosing of the drug held within well 3.

The focal point 8 of the parabolic surface 7 defines a point of maximum energy. Focussing of the ultrasonic energy provides a more efficient nebulisation process and a process that can be more precisely controlled compared to prior art devices. Furthermore, because of the focussing, energy requirements to drive the nebuliser are comparatively lower, meaning that the nebuliser can be reduced in overall dimensions compared to prior art devices. The applicant has further surprisingly found the time to form an acceptable fountain 12 of nebulised liquid 3 is reduced compared to prior art devices, e.g. less than 0.1 second. This may be due to the relatively reduced volume of fluid which is absorbing the energy as compared to conventional nebulisers. Further still, focussing the ultrasonic energy into a relatively shallow pool of drug 4 allows for most of the drug to be nebulised and delivered to the patient. Consequently, the nebuliser of the invention provides for reduced drug wastage compared to prior art devices.

As would be understood by persons skilled in the art, during formation of the vertically extending liquid fountain 12 rising from well 2, the upper portion of the fountain 12 can, in a conventional construction fall down upon itself thereby reducing the energy of the fountain 12. This requires additional energy to be provided by energy source 6 and in some cases can reduce efficient nebulisation of the liquid 3 in well 3.

In an effort to overcome some of these disadvantages, the nebuliser provides a deflector baffle 16 positioned within the nebulisation chamber directly above the well 2. The deflector baffle 16 is adapted to deflect the liquid fountain 12 rising from the well 2 in a direction away from the axis of the fountain. Preferably, the liquid fountain 12 is deflected substantially to one side of its axis. Desirably, the deflector baffle 16 is placed intermediate the well 2 and the “unhindered apex” of the liquid fountain 12. The term “unhindered apex” of the liquid fountain refers to the height of the liquid fountain if the deflector baffle 16 was not in place.

In a preferred embodiment, the deflector baffle 16 is substantially in the shape of an inverted U-shaped tube in which the redirected fountain 12, or any condensate 17, is transferred to a bank 18 of the well 2 which is inclined to promote recirculation of the liquid 3. The redirected fountain reduces interference of the focal point 9 by any condensed liquid 17, or the returning fountain itself. In the preferred embodiment, the apex 19 of the fountain diverter 16 is spaced from the axis of the fountain 12. The inverted U-shaped tube optionally includes a deflection surface 20 wherein the fountain directly impinges on the deflection surface 20 during nebulisation.

It will be clear to persons skilled in the art that the arrangement of such a deflector baffle 16 has significant advantages over the prior art. The deflection of the fountain prior to it unhindered apex reduces the possibility of the fountain falling back on itself thereby reducing its height and energy. In addition, deflection of the fountain or indeed other liquid 3 arising from the well 2 assists in recirculation of the liquid 3 back to the well 2 for subsequent nebulisation. This is particularly important in the present invention which preferably includes a well 2 which has relatively small quantity of liquid 3 contained therein. In such an instance it is important that any non-nebulised liquid 3 be returned to the well 2 as rapidly as possible for subsequent nebulisation to ensure an accurate continuous dosage of the liquid 3 is provided while the energy source 6 is actuated.

Referring again to the drawings the nebulisation chamber 1 defines a circuitous path between the well 2 and the aerosol outlet 14. This circuitous or labyrinthine path is provided by at least one baffle 21 mounted within the nebulisation chamber 1. The Applicant has surprisingly found that this circuitous flow path permits aerosol 13 to be transported to the patient but non-aerosolised liquid 17 to be returned to the well 2 for recycling and further nebulisation.

Unlike conventional nebulisers the circuitous path provided by nebulisation chamber 1 assists in inhalation of the nebulised liquid and recirculation of non-nebulised liquid 3 to the well 2.

To explain, the nebulised liquid effectively has a “neutral buoyancy”. In other words it simply floats in air. Therefore this nebulised liquid can proceed along the circuitous path in the nebulisation chamber toward outlet 14. Non-nebulised liquid, however, is drawn along the circuitous path by means of the negative pressure applied to outlet 14 and generally impacts 1 or more of the baffles in the nebulisation chamber. This non-nebulised liquid or large droplets are not only ineffective in terms of drug delivery but due to the highly efficient low dosage arrangement of the present invention they can significantly impact on the quantity of liquid remaining in the well and thereby negatively impact on subsequent dosages. Accordingly the circuitous path has the additional benefit of “collecting” and returning the aforementioned non-nebulised liquid toward well 2 for subsequent nebulisation.

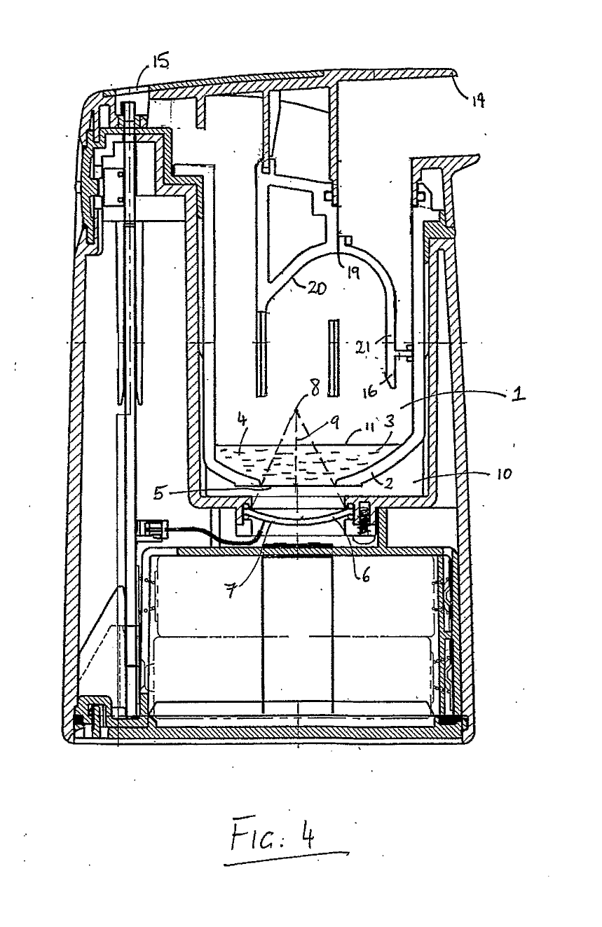

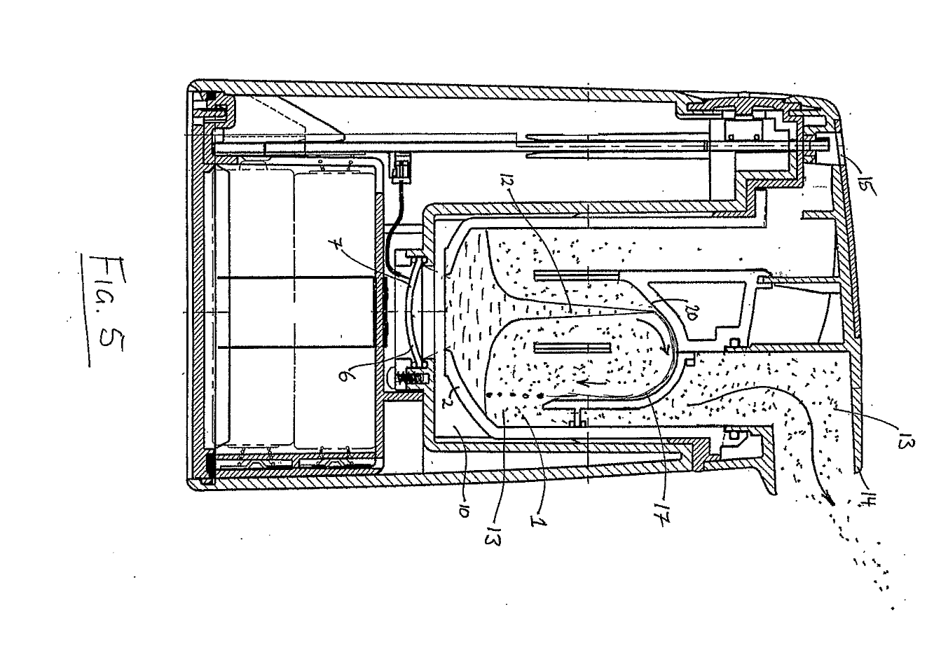

Turning now to the second nebuliser embodiment as shown in FIGS. 4 and 5, like features have been given like reference numerals. In this embodiment the ultrasonic transducer 6 is spaced from the well 2 such that the focal point 8 is positioned above the surface 11 of the nebulisable liquid 3. Preferably the distance between the focal point 8 and the surface 11 is not greater than 50% of the focal length 9. As shown in FIGS. 4 and 5, the ultrasonic transducer 6 is disposed directly beneath the well 2 and the focal point 8 is positioned above the surface 11 of the nebulisable liquid 3 when the nebuliser is held in a substantially upright position.

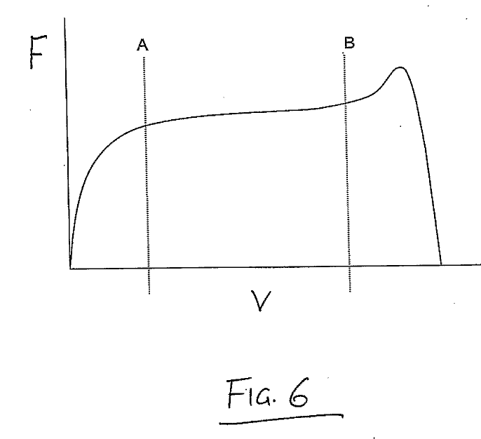

The well 2 is shaped such that during nebulisation the level of nebulisable liquid 4 remains within a predetermined focal length range to thereby provide a substantially constant flow rate of nebulised liquid. Preferably the predetermined focal length range is such that flow rate remains within 10% of maximum flow rate. FIG. 6 shows the flow rate of nebulised drug versus the drug height remaining in the well. The lines marked as A and B correspond to the lower and upper respectively focal length ranges at which the flow rate of nebulised drug remains substantially constant. The drug well is shaped such that the drug height/volume remaining in the well at points A and B correspond to the lower and upper respectively focal length ranges. Preferably the volume of drug contained in the well at A and B is 1 and 6 mL respectively. If the device is charged with liquid to a height equal to the focal point 8, the flow rate increases to a maximum, as shown in FIG. 6. If further liquid is added, making the focal point below the surface of the liquid, the flow rate is less than maximum. If there is insufficient liquid in the well, i.e. the liquid level is below point A, there is insufficient liquid to form a suitable fountain of nebulised liquid and the flow rate reduces sharply. Preferably the well includes an inverted frusto-conical bottom wall disposed about a base portion and the bottom wall forms a liquid reservoir that drains towards the base portion.

In one embodiment nebulisation has proved very effective. For instance the average particle size of the aerosol 13 formed by the nebuliser has been measured by suitable optical techniques at less than 5 micron and the aerosol flow rate measured at up to about 0.8 mL/min.

As discussed above, the nebuliser is particularly suitable for use where high concentration low dosage drugs are to be delivered e.g. in the area of sexual dysfunction in men and women.

In the treatment of sexual dysfunction in men and women, injections, suppositories, lozenges, transdermal patches, tablets and intra-urethral pellets, creams and gels are typically prescribed. These routes of administration typically require up to 100 mg or more of active ingredient in each dose to be therapeutically effective. This is because bioavailability is relatively low from these routes of administration. Nasal sprays are a recently emerging technology for the treatment of sexual dysfunction in men and women. While these use lower dosages than the aforementioned conventional treatments they still require higher quantities than the proposed new method and device. In contrast, pulmonary administration of an aerosol of drug provides a relatively faster and higher bioavailability. For example, it has been estimated that pulmonary inhalation of 5 mg of a drug such as sildenafil provides an equivalent therapeutic effect compared to a 50 mg tablet but in a fraction of the time. Relatively less drug is required for pulmonary administration as less is wasted compared to these other administration routes.

The nebuliser can be configured to deliver a dose of about 5 mg of a drug, which the Applicant estimates to be greater than 90% bioavailablity. For example, 5 mg of a drug such as sildenafil can be delivered by pulmonary inhalation by a 1.5 second burst of nebulisation of a sildenafil solution having 100 mg/mL concentration and with an aerosol flow rate of 0.8 mL/min. The nebuliser can be configured to provide a range of doses by varying the drug concentration and varying the time that the ultrasonic transducer 6 is energised for. For example, doses between about 0.1 to 50 mg of drug are achievable. Due to the volume of drug contained in the well, and the precise control over the nebulisation time, the nebuliser may also be considered to be a “multi-dose” device.

The nebuliser of the invention can surprisingly provide a clinically effective treatment in less than 10 seconds. Furthermore, pulmonary administration of a drug such as sildenafil using the nebuliser of the invention may provide an onset of therapeutic effect in less than about 10 minutes.

In a particular embodiment the nebuliser operates on 4 x 1.2 volt batteries 24 connected in series to provide a total of 4.8 Volts and 1600 milliamp-hours. The ultrasonic transducer 6 delivers 5-6 Watts at 2-6 MHz. The power requirement is relatively small because no fan is required to pump the aerosol 13 to the patient and the device is “on-demand”.

It will be appreciated that the illustrated nebuliser is effective, economical and convenient and simple to use. The nebuliser generates a relatively large amount of aerosol in a relatively short time period and having a reproducible predetermined particle size range.

Although the invention has been described with reference to specific examples, it will be appreciated by those skilled in the art that the invention may be embodied in many other forms.

1-173. (canceled)

174. A nebuliser comprising:
a nebulisation chamber having a well adapted to contain a nebulisable liquid;
an energy source operatively associated with said well to nebulise said nebulisable liquid, said energy source having a curved energy transmission surface thereby defining a focal point and focal length of energy produced by said source; and
wherein said energy source is spaced from said well such that said focal point is positioned above the surface of said nebulisable liquid and the distance between said focal point and said energy source intrudes into said well not greater than 50% of said focal length.

175. A nebuliser according to claim 174 wherein said energy source is an ultrasonic transducer.

176. A nebuliser according to claim 174 wherein said curved energy transmission surface is parabolic.

177. A nebuliser according to claim 174 wherein said well is disposed at substantially the deepest part of said nebulisation chamber.

178. A nebuliser according to claim 174 wherein said well is adapted to contain a relatively shallow layer of nebulisable liquid.

179. A nebuliser according to claim 174 wherein actuation of said energy source produces a fountain of nebulised liquid, said nebulised liquid having a particle size below a predetermined particle size.

180. A nebuliser according to claim 179 wherein said predetermined particle size is 5 micron.

181. A nebuliser according to claim 179 wherein said predetermined particle size is that which provides the nebulised liquid droplet with neutral buoyancy.

182. A nebuliser according to claim 174 wherein said well contains up to 8 mL of a nebulisable liquid.

183. A nebuliser according to claim 174 wherein the distance between said focal point and the surface of the nebulisable liquid is not greater than 50% of said focal length.

184. A method of nebulising a nebulisable liquid comprising:
containing said nebulisable liquid in a well;
providing an energy source having a curved energy transmission surface defining a focal point and focal length of energy produced by said source;
transmitting energy from said source to said well to thereby nebulise said nebulisable liquid contained therein;
wherein said energy source is spaced from said well such that said focal point is positioned above the surface of said nebulisable liquid and the distance between said focal point and said energy source intrudes into said well not greater than 50% of said focal length.

185. A method according to claim 184 wherein said energy source is an ultrasonic transducer.

186. A method according to claim 184 wherein said curved energy transmission surface is parabolic.

187. A method according to claim 184 wherein said nebulisable liquid is chosen from the group consisting of: a drug, a solution of a drug and a suspension of a drug.

188. A method according to claim 184 wherein said well is adapted to contain a relatively shallow layer of nebulisable liquid.

189. A method according to claim 184 wherein said well is housed in a chamber having an exit to allow egress of said nebulised liquid.

190. A method according to claim 189 further including the step of drawing said nebulised liquid from said chamber through said exit.

191. A method according to claim 184 wherein actuation of said energy source produces a fountain of nebulised liquid, said nebulised liquid having a particle size below a predetermined particle size.

192. A method according to claim 191 wherein said predetermined particle size is that which provides the nebulised liquid droplet substantially with neutral buoyancy.

193. A method according to claim 184 wherein the distance between said focal point and the surface of the nebulisable liquid is not greater than 50% of said focal length.

194. A nebuliser comprising:
a nebulisation chamber having a well adapted to contain a nebulisable liquid;
an energy source operatively associated with said well to nebulise said nebulisable liquid and thereby produce a fountain of nebulised liquid rising from said well; and
a deflector baffle positioned directly above said well and adapted to deflect said nebulised liquid fountain rising from said well, wherein said deflector baffle is shaped as an inverted U and the apex of said inverted U is spaced from an axis of said fountain, said inverted U including a substantially planar deflection surface adjacent its apex and wherein said fountain impinges on said deflection surface during nebulisation.

195. A nebuliser according to claim 194 wherein said energy source includes a curved energy transmission surface.

196. A nebuliser according to claim 194 wherein said deflector baffle is positioned to deflect substantially all liquid that impinges upon said deflector baffle away from the axis of said liquid fountain.

197. A nebuliser according to claim 196 wherein said deflector baffle is positioned to deflect substantially all liquid that impinges upon said deflector baffle in at least one direction away from the axis of said liquid fountain.

198. A nebuliser according to claim 196 wherein said fountain of said nebulised liquid is deflected such that the fountain does not fall back on itself.

199. A nebuliser according to claim 198 wherein any deflected liquid is returned to said well for re-nebulisation.

200. A nebuliser according to claim 194 wherein actuation of said energy source produces a fountain of nebulised liquid, said nebulised liquid having a particle size below a predetermined particle size.

201. A nebuliser according to claim 200 wherein said predetermined particle size is 5 micron.

202. A nebuliser according to claim 200 wherein said predetermined particle size is that which provides the nebulised liquid droplet with neutral buoyancy.

203. A method of nebulising a nebulisable liquid in a nebuliser, said nebuliser having a nebulisation chamber including a well adapted to contain a nebulisable liquid and an energy source operatively associated with the well to nebulise said nebulisable liquid, said method comprising:
providing a deflector baffle directly above said well;
forming a liquid fountain by nebulising said nebulisable liquid; and
deflecting said liquid fountain rising from said well,
wherein said deflector baffle is shaped as an inverted U and the apex of said inverted U is spaced from an axis of said fountain, said inverted U including a substantially planar deflection surface adjacent its apex, wherein said fountain impinges on said deflection surface during nebulisation.

204. A method according to claim 203 wherein said energy source is an ultrasonic transducer.

205. A method according to claim 203 wherein said energy source includes a curved energy transmission surface.

206. A method according to claim 203 wherein said nebulisable liquid is chosen from the group consisting of: a drug, a solution of a drug and a suspension of a drug.

207. A method according to claim 203 wherein said deflector baffle is positioned to deflect substantially all liquid that impinges upon said deflector baffle away from the axis of said liquid fountain.

208. A method according to claim 207 wherein said fountain of said nebulised liquid is deflected such that the fountain does not fall back on itself.

209. A method according to claim 203 further including the step of returning to said well any deflected liquid for re-nebulisation.

210. A method according to claim 203 wherein actuation of said energy source produces a fountain of nebulised liquid, said nebulised liquid having a particle size below a predetermined particle size.

211. A method according to claim 210 wherein said predetermined particle size is 5 micron.

212. A method according to claim 210 wherein said predetermined particle size is that which provides the nebulised liquid droplet with substantially neutral buoyancy.

213. A nebuliser comprising:
a nebulisation chamber having a well adapted to contain a nebulisable liquid; and
an energy source spaced from and operatively associated with said well to nebulise said nebulisable liquid, said energy source including a curved energy transmission surface thereby defining an energy focal point and a focal length between said energy source and said focal point, wherein said focal point is positioned above the surface of said nebulisable liquid,
said well being shaped such that during nebulisation the level of nebulisable liquid remains within a predetermined focal length range to thereby provide a substantially constant flow rate of nebulised liquid.

214. A nebuliser according to claim 213 wherein said curved energy transmission surface is parabolic.

215. A nebuliser according to claim 213 wherein actuation of said energy source produces a fountain of nebulised liquid, said nebulised liquid having a particle size below a predetermined particle size.

216. A nebuliser according to claim 215 wherein said predetermined particle size is 5 micron.

217. A nebuliser according to claim 213 wherein said predetermined particle size is that which provides the nebulised liquid droplet with neutral buoyancy.

218. A nebuliser according to claim 213 wherein said predetermined focal length range is such that said flow rate remains within 10% of a maximum flow rate.

219. A nebuliser according to claim 218 wherein said maximum flow rate is 1.5 mL/min.

220. A nebuliser according to claim 218 wherein said maximum flow rate is between 0.8 and 1.2 mL/min.

221. A nebuliser according to claim 213 wherein said well contains up to 8 mL of a nebulisable liquid.

222. A nebuliser according to claim 221 wherein said predetermined focal length range providing said substantially constant flow rate of nebulised liquid corresponds to a volume contained in said well of between 1 and 6 mL.

223. A method of nebulising a nebulisable liquid comprising:
providing a nebulisation chamber having a well adapted to contain a nebulisable liquid;
providing an energy source spaced from and operatively associated with said well to nebulise said liquid, said energy source including a curved energy transmission surface thereby defining an energy focal point and a focal length between said energy source and said focal point, wherein said focal point is positioned above the surface of said nebulisable liquid; and
actuating said energy source to nebulise said nebulisable liquid, such that during nebulisation the level of liquid in said well remains within a predetermined focal length range thereby providing a substantially constant flow rate of nebulised liquid.

224. A method according to claim 223 wherein actuation of said energy source produces a fountain of nebulised liquid, said nebulised liquid having a particle size below a predetermined particle size.

225. A method according to claim 224 wherein said predetermined particle size is 5 micron.

226. A method according to claim 224 wherein said predetermined particle size is that which provides the nebulised liquid droplet substantially with neutral buoyancy.

227. A method according to claim 223 wherein said well is shaped such that during nebulisation the level of liquid in said well remains within a predetermined focal length range thereby providing a substantially constant flow rate of nebulised liquid.

228. A method according to claim 227 wherein said predetermined focal length range is such that said flow rate remains within 10% of a maximum flow rate.

229. A method according to claim 228 wherein said maximum flow rate is 1.5 mL/min.

230. A method according to claim 228 wherein said maximum flow rate is between 0.8 and 1.2 mL/min.

231. A method according to 223 wherein up to 8 mL of a nebulisable liquid is contained in said well.

232. A method according to claim 231 wherein said predetermined focal length range providing said substantially constant flow rate of nebulised liquid corresponds to a volume contained in said well of between 1 and 6 mL of said liquid.
